(19) United States
(12) Patent Application Publication
(10) Pub. No.: US 2022/0033882 A1
(43) Pub. Date: Feb. 3, 2022

(54) METHODS OF DIAGNOSING AND TREATING PATIENTS WITH PIGMENTED SKIN LESIONS

(71) Applicant: CASTLE BIOSCIENCES, INC., Friendswood, TX (US)

(72) Inventors: Robert Willis COOK, Houston, TX (US); Derek MAETZOLD, Friendswood, TX (US); Kyle R. COVINGTON, Friendswood, TX (US); Olga ZOLOCHEVSKA, Friendswood, TX (US)

(73) Assignee: CASTLE BIOSCIENCES, INC., Friendswood, TX (US)

(21) Appl. No.: 17/392,706

(22) Filed: Aug. 3, 2021

Related U.S. Application Data
(60) Provisional application No. 63/084,520, filed on Sep. 28, 2020.
(60) Provisional application No. 63/060,661, filed on Aug. 3, 2020.

(51) Int. Cl. C12Q 1/6809; C12Q 1/6806; G16B 5/20; G16B 40/00

(52) U.S. Cl. CPC C12Q 1/6809; C12Q 1/6806; C12Q 2600/158; G16B 40/00; C12Q 2600/166; G16B 5/20

(57) ABSTRACT

The present disclosure relates to methods for diagnosing a skin lesion as malignant or benign.

### CROSS REFERENCE TO RELATED APPLICATIONS

This application claims the benefit of U.S. Provisional Patent Application No. 63/060,661, filed Aug. 3, 2020, and U.S. Provisional Patent Application No. 63/084,520, filed Sep. 28, 2020, the disclosures of each of which are incorporated by reference in their entirety.

### FIELD OF THE DISCLOSURE

The present disclosure relates to methods for diagnosing and/or treating patients with suspicious pigmented skin lesions.

### BACKGROUND

Over 5 million skin biopsies are performed annually in the United States, leading to diagnosis of over 130,000 invasive melanoma tumors. Because melanoma is the most aggressive skin cancer, early detection and diagnosis are crucial. Current methods used for definitive diagnosis of melanoma are sufficient for the majority of lesions; however, histopathologic assessment can be challenging, even for experienced dermatopathologists, and high rates of diagnostic discordance have been reported. Even pigmented lesions with clear pathological features consistent with benign nevi or invasive melanoma have concordance rates of 92% and 72%, respectively, indicating that a subset of lesions with typical histopathological presentation are subject to differential assessment. Visual assessment of haematoxylin and eosin (H&E) stained lesions is inherently subjective and relies on expert interpretation of a wide spectrum of cellular presentations including, but not limited to normal, gradients of atypia, and various cancer types where thresholds for diagnosing a benign or malignant lesion vary depending on training and experience of a dermatopathologist. For instance, certain melanoma subtypes such as desmoplastic, Spitzoid, nevoid, lentigo maligna, and lentiginous all have a differential diagnosis of nevi subtypes with similar histologic properties often making the distinction of melanoma dependent on additional evaluations. Difficult-to-diagnose lesions are commonly sent for second opinions to dermatopathologists who have more experience with atypical cases. Ambiguous lesions represent a subset of pigmented lesions that are difficult to diagnose leading to reported discordant rates of 25-43%. Studies detailing the prevalence, outcome, and misdiagnosis of these lesions indicate that improved ancillary diagnostic technologies could be beneficial to the dermatopathologist and dermatologist in determining the most appropriate treatment plan.

Efforts to improve melanoma diagnosis have traditionally focused on ancillary tests such as immunohistochemistry (IHC), fluorescence in situ hybridization (FISH), and comparative genomic hybridization (CGH), but each has limitations. FISH and CGH can have suboptimal sensitivity and specificity in some subtypes of pigmented lesions and are not used as widely as IHC. IHC is the most commonly utilized diagnostic tool for melanocytic lesions, but IHC to identify S100, Ki-67, MelanA and other markers is limited by subjective interpretation of protein expression levels. Similarly, a recently developed PRAIVIE IHC assay has exhibited staining patterns in some nevi (˜14%) that are above the threshold established for a diagnosis of melanoma.

Definitive diagnosis is also complicated for a subset of lesions described as being borderline, indeterminant, of unknown malignant potential (UMP), atypical melanocytic proliferation (AMP) or in a ‘grey’ zone. Clinical management of these cases usually results in conservative treatment for the “most significant consideration in the differential diagnosis”. GEP has been employed to improve the diagnosis of these suspicious pigmented lesions. While a 2-gene pigmented lesion array (2-gene) and a 23-gene expression profile (GEP) test have been developed, the 2-gene utility is focused on guiding biopsy decisions by dermatologists; whereas, the 23-GEP labels a substantial number of lesions (˜15% across studies) as indeterminate rather than providing a result of benign or malignant. Approximately 10% of unequivocal cases and 15% of ambiguous lesions may be labeled indeterminate by the 23-GEP test, and although sensitivity and specificity is reported at 91.5% and 92.5%, respectively, there is an opportunity to increase accuracy to optimize the clinical diagnosis of melanoma, particularly given the advances in melanoma prognosis and treatment over the past decade.

To address the need for more accurate predictive factors, for difficult-to-diagnose lesions in particular, and facilitate appropriate intervention strategies, gene expression analysis was used to differentiate between benign and malignant pigmented lesions.

### SUMMARY

There is a need in the art for a more accurate method of differentiating between benign and malignant pigmented skin lesions. Disclosed herein is the development and validation of a 35-GEP test to differentiate between benign and malignant pigmented lesions with greater accuracy than previously developed tests. A training cohort of samples, including subtypes considered challenging to diagnose, was established and bioinformatic and machine-learning approaches were used to select and prioritize genes associated with benign or malignant biology. The test was validated using an independent cohort of cases and demonstrates sensitivity and specificity metrics exceeding those currently reported in the melanoma diagnostic literature while maintaining a minimal indeterminate-risk zone. The novel 35-GEP test could aid in the diagnosis of suspicious pigmented lesions and improve accuracy alone or when used in combination with currently applied diagnostic tools.

In one embodiment, the disclosure provides a method for treating a patient with a skin lesion, the method comprising:

(a) obtaining a diagnosis identifying the skin lesion as a malignant lesion, an intermediate risk lesion, or a benign lesion in a sample from the skin lesion of the patient, wherein the diagnosis was obtained by:
(1) determining the expression level of at least 12 genes in a gene set; wherein the at least 12 genes in the gene set are selected from:
ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1;
(2) comparing the expression levels of the at least 12 genes in the gene set from the sample to the expression levels of the at least 12 genes in the gene set from a predictive training set to generate a probability score;
(3) providing an indication as to whether the skin lesion is a malignant lesion, an intermediate risk lesion, or a benign lesion based on the probability score generated in step (2); and
(4) identifying that the skin lesion is a malignant lesion based on the probability score; and

(b) administering to the patient an aggressive treatment when the determination is made in the affirmative that the patient has a skin lesion that is a malignant lesion.

In certain embodiments the gene set comprises ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1.

In some embodiments, the method further comprises performing a resection of the skin lesion when the determination is made in the affirmative that the patient has a skin lesion that is a malignant lesion.

In certain embodiments of the method, the expression level of each gene in a gene set is determined by reverse transcribing the isolated mRNA and measuring a level of fluorescence for each gene in the gene set by a nucleic acid sequence detection system following RT-PCR.

In certain embodiments, the sample from the skin lesion is obtained from a formalin-fixed, paraffin embedded sample.

In certain embodiments, the probability score is between 0 and 1, and wherein a value of 1 indicates a higher probability of a malignant lesion than a value of 0.

In certain embodiments, the probability score has a sensitivity of at least 90%, 91%, 92%, 93%, 94%, 95%, 96%, 97%, 98%, or 99%, and/or has a specificity of at least 90%, 91%, 92%, 93%, 94%, 95%, 96%, 97%, 98%, or 99%.

In certain embodiments, the probability score has a positive predictive value (PPV) of at least 90%, 91%, 92%, 93%, 94%, 95%, 96%, 97%, 98%, or 99%, and/or has a negative predictive value (NPV) of at least 90%, 91%, 92%, 93%, 94%, 95%, 96%, 97%, 98%, or 99%.

In certain embodiments, the gene set further comprises at least one control gene, wherein the at least one control gene is selected from the group consisting of FXR1, HNRNPL, and YKT6. In some embodiments, the control genes are FXR1, HNRNPL, and YKT6.

In another embodiment, the disclosure provides a method of treating a patient with a skin lesion, the method comprising administering an aggressive cancer treatment regimen to the patient,

wherein the patient has a skin lesion identified as a malignant lesion as generated by comparing the expression levels of at least 12 genes wherein the at least 12 genes are selected from ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1, from the skin lesion with the expression levels of the same at least 12 genes are selected from ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1 from a predictive training set.

In certain embodiments the gene set comprises ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1.

In certain embodiments, the skin lesion is determined to be a malignant lesion, an intermediate risk lesion, or a benign lesion.

In certain embodiments, the gene set further comprises at least one control gene, wherein the at least one control gene is selected from the group consisting of FXR1, HNRNPL, and YKT6. In some embodiments, the control genes are FXR1, HNRNPL, and YKT6.

In yet another embodiment, the disclosure provides a kit comprising primer pairs suitable for the detection and quantification of nucleic acid expression of at least 12 genes, wherein the at least 12 genes are selected from: ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1. In certain embodiments the kit comprises primer pairs for each of ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1.

In certain embodiments, the kit further comprises primer pairs suitable for the detection and quantification of nucleic acid expression of at least one control gene, wherein the at least one control gene is selected from the group consisting of FXR1, HNRNPL, and YKT6. In some embodiments, the kit comprises primer pairs for 3 control genes, wherein the 3 control genes are FXR1, HNRNPL, and YKT6.

In another embodiment, the disclosure provides a method for diagnosing a skin lesion from a patient as a malignant lesion, an intermediate risk lesion, or a benign lesion, the method comprising:

(a) obtaining a sample from the skin lesion from the patient and isolating mRNA from the sample;

(b) determining the expression level of at least 12 genes in a gene set; wherein the at least 12 genes in the gene set are selected from: ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1;

(c) comparing the expression levels of the at least 12 genes in the gene set from the sample to the expression levels of the at least 12 genes in the gene set from a predictive training set to generate a probability score; and

(d) providing an indication as to whether the skin lesion is a malignant lesion, an intermediate risk lesion, or a benign lesion, based on the probability score generated in step (c).

In certain embodiments the gene set comprises ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1.

In certain embodiments, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR). In some embodiments, the sample from the skin lesion is obtained from a formalin-fixed, paraffin embedded sample.

In certain embodiments, the probability score is between 0 and 1, and wherein a value of 1 indicates a higher probability of a malignant lesion than a value of 0.

In certain embodiments, the probability score has a sensitivity of at least 90%, 91%, 92%, 93%, 94%, 95%, 96%, 97%, 98%, or 99%, and/or has a specificity of at least 90%, 91%, 92%, 93%, 94%, 95%, 96%, 97%, 98%, or 99%.

In certain embodiments, the probability score has a positive predictive value (PPV) of at least 90%, 91%, 92%, 93%, 94%, 95%, 96%, 97%, 98%, or 99%, and/or has a negative predictive value (NPV) of at least 90%, 91%, 92%, 93%, 94%, 95%, 96%, 97%, 98%, or 99%.

In certain embodiments, the method further comprises identifying the skin lesion as a malignant lesion based on the probability score, and administering to the patient an aggressive tumor treatment.

In certain embodiments, the gene set further comprises at least one control gene, wherein the at least one control gene is selected from the group consisting of FXR1, HNRNPL, and YKT6. In some embodiments, the control genes are FXR1, HNRNPL, and YKT6.

In another embodiment, the disclosure provides a method for diagnosing a skin lesion from a patient as a malignant lesion, an intermediate risk lesion, or a benign lesion, the method comprising:

(a) obtaining a sample from skin lesion from the patient and isolating mRNA from the sample;

(b) determining the expression level of at least 12 genes in a gene set; wherein the at least 12 genes in the gene set are: ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1; and

(c) providing an indication as to whether the skin lesion is a malignant lesion, an intermediate risk lesion, or a benign lesion, based on the expression level of the at least 12 genes generated in step (b).

In certain embodiments the gene set comprises ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1.

In certain embodiments, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR). In some embodiments, the sample from the skin lesion is obtained from formalin-fixed, paraffin embedded sample.

In certain embodiments, the gene set further comprises at least one control gene, wherein the at least one control gene is selected from the group consisting of FXR1, HNRNPL, and YKT6. In some embodiments, the control genes are FXR1, HNRNPL, and YKT6.

Other aspects, embodiments, and implementations will become apparent from the following detailed description and claims, with reference, where appropriate, to the accompanying drawings.

### BRIEF DESCRIPTION OF THE DRAWINGS

FIG. 1 shows the study cohorts. Study was performed on 498 benign and 453 malignant lesions accrued under an IRB-approved protocol in a multicenter cohort. Thirty-two samples (˜3.4%) of the Study Cohort were excluded from further analysis due to MGF with the remaining 919 samples randomized to training or validation cohorts while conserving benign or melanoma subtype representation in each cohort. Training (200 benign nevi and 216 melanomas) and validation (273 benign nevi and 230 melanomas) cohorts' demographic details are shown in Table 1. *MGF (multiple gene failure) rules: control genes were evaluated independently and failure of any control gene resulted in sample exclusion. Triplicate gene expression data were aggregated and normalized using control probes. Samples with failure to amplify ≥3 out of 73 genes were excluded from model development and validation.

### DETAILED DESCRIPTION

There is a need in the art for a more accurate method of differentiating between benign and malignant pigmented skin lesions. Disclosed herein is the development and validation of a 35-GEP test to differentiate between benign and malignant pigmented lesions with greater accuracy than previously developed tests. A training cohort of samples, including subtypes considered challenging to diagnose, was established and bioinformatic and machine-learning approaches were used to select and prioritize genes associated with benign or malignant biology. The test was validated using an independent cohort of cases and demonstrates sensitivity and specificity metrics exceeding those currently reported in the melanoma diagnostic literature while maintaining a minimal indeterminate-risk zone. The novel 35-GEP test could aid in the diagnosis of suspicious pigmented lesions and improve accuracy alone or when used in combination with currently applied diagnostic tools.

In one embodiment, the disclosure provides a method for treating a patient with a skin lesion, the method comprising:

(a) obtaining a diagnosis identifying the skin lesion as a malignant lesion, an intermediate risk lesion, or a benign lesion in a sample from the skin lesion of the patient, wherein the diagnosis was obtained by:
(1) determining the expression level of at least 12 genes in a gene set; wherein the at least 12 genes in the gene set are selected from: ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1;
(2) comparing the expression levels of the at least 12 genes in the gene set from the sample to the expression levels of the at least 12 genes in the gene set from a predictive training set to generate a probability score;
(3) providing an indication as to whether the skin lesion is a malignant lesion, an intermediate risk lesion, or a benign lesion based on the probability score generated in step (2); and
(4) identifying that the skin lesion is a malignant lesion based on the probability score; and

(b) administering to the patient an aggressive treatment when the determination is made in the affirmative that the patient has a skin lesion that is a malignant lesion.

In certain embodiments the gene set comprises ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1.

In some embodiments, the method further comprises performing a resection of the skin lesion when the determination is made in the affirmative that the patient has a skin lesion that is a malignant lesion.

In certain embodiments of the method, the expression level of each gene in a gene set is determined by reverse transcribing the isolated mRNA and measuring a level of fluorescence for each gene in the gene set by a nucleic acid sequence detection system following RT-PCR.

In certain embodiments, the sample from the skin lesion is obtained from a formalin-fixed, paraffin embedded sample.

In certain embodiments, the probability score is between 0 and 1, and wherein a value of 1 indicates a higher probability of a malignant lesion than a value of 0.

In certain embodiments, the probability score has a sensitivity of at least 90%, 91%, 92%, 93%, 94%, 95%, 96%, 97%, 98%, or 99%, and/or has a specificity of at least 90%, 91%, 92%, 93%, 94%, 95%, 96%, 97%, 98%, or 99%.

In certain embodiments, the probability score has a positive predictive value (PPV) of at least 90%, 91%, 92%, 93%, 94%, 95%, 96%, 97%, 98%, or 99%, and/or has a negative predictive value (NPV) of at least 90%, 91%, 92%, 93%, 94%, 95%, 96%, 97%, 98%, or 99%.

In certain embodiments, the gene set further comprises at least one control gene, wherein the at least one control gene is selected from the group consisting of FXR1, HNRNPL, and YKT6. In some embodiments, the control genes are FXR1, HNRNPL, and YKT6.

In another embodiment, the disclosure provides a method of treating a patient with a skin lesion, the method comprising administering an aggressive cancer treatment regimen to the patient, wherein the patient has a skin lesion identified as a malignant lesion as generated by comparing the expression levels of at least 12 genes wherein the at least 12 genes are selected from ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1, from the skin lesion with the expression levels of the same at least 12 genes are selected from ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1 from a predictive training set.

In certain embodiments the gene set comprises ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1.

In certain embodiments, the skin lesion is determined to be a malignant lesion, an intermediate risk lesion, or a benign lesion.

In certain embodiments, the gene set further comprises at least one control gene, wherein the at least one control gene is selected from the group consisting of FXR1, HNRNPL, and YKT6. In some embodiments, the control genes are FXR1, HNRNPL, and YKT6.

In yet another embodiment, the disclosure provides a kit comprising primer pairs suitable for the detection and quantification of nucleic acid expression of at least 12 genes, wherein the at least 12 genes are selected from: ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1. In certain embodiments the kit comprises primer pairs for each of ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1.

In certain embodiments, the kit further comprises primer pairs suitable for the detection and quantification of nucleic acid expression of at least one control gene, wherein the at least one control gene is selected from the group consisting of FXR1, HNRNPL, and YKT6. In some embodiments, the kit comprises primer pairs for 3 control genes, wherein the 3 control genes are FXR1, HNRNPL, and YKT6.

In another embodiment, the disclosure provides a method for diagnosing a skin lesion from a patient as a malignant lesion, an intermediate risk lesion, or a benign lesion, the method comprising:

(a) obtaining a sample from the skin lesion from the patient and isolating mRNA from the sample;

(b) determining the expression level of at least 12 genes in a gene set; wherein the at least 12 genes in the gene set are selected from: ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1;

(c) comparing the expression levels of the at least 12 genes in the gene set from the sample to the expression levels of the at least 12 genes in the gene set from a predictive training set to generate a probability score; and

(d) providing an indication as to whether the skin lesion is a malignant lesion, an intermediate risk lesion, or a benign lesion, based on the probability score generated in step (c).

In certain embodiments the gene set comprises ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1.

In certain embodiments, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR). In some embodiments, the sample from the skin lesion is obtained from a formalin-fixed, paraffin embedded sample.

In certain embodiments, the probability score is between 0 and 1, and wherein a value of 1 indicates a higher probability of a malignant lesion than a value of 0.

In certain embodiments, the probability score has a sensitivity of at least 90%, 91%, 92%, 93%, 94%, 95%, 96%, 97%, 98%, or 99%, and/or has a specificity of at least 90%, 91%, 92%, 93%, 94%, 95%, 96%, 97%, 98%, or 99%.

In certain embodiments, the probability score has a positive predictive value (PPV) of at least 90%, 91%, 92%, 93%, 94%, 95%, 96%, 97%, 98%, or 99%, and/or has a negative predictive value (NPV) of at least 90%, 91%, 92%, 93%, 94%, 95%, 96%, 97%, 98%, or 99%.

In certain embodiments, the method further comprises identifying the skin lesion as a malignant lesion based on the probability score, and administering to the patient an aggressive tumor treatment.

In certain embodiments, the gene set further comprises at least one control gene, wherein the at least one control gene is selected from the group consisting of FXR1, HNRNPL, and YKT6. In some embodiments, the control genes are FXR1, HNRNPL, and YKT6.

In another embodiment, the disclosure provides a method for diagnosing a skin lesion from a patient as a malignant lesion, an intermediate risk lesion, or a benign lesion, the method comprising:

(a) obtaining a sample from skin lesion from the patient and isolating mRNA from the sample;

(b) determining the expression level of at least 12 genes in a gene set; wherein the at least 12 genes in the gene set are: ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1; and

(c) providing an indication as to whether the skin lesion is a malignant lesion, an intermediate risk lesion, or a benign lesion, based on the expression level of the at least 12 genes generated in step (b).

In certain embodiments the gene set comprises ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1.

In certain embodiments, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR). In some embodiments, the sample from the skin lesion is obtained from formalin-fixed, paraffin embedded sample.

In certain embodiments, the gene set further comprises at least one control gene, wherein the at least one control gene is selected from the group consisting of FXR1, HNRNPL, and YKT6. In some embodiments, the control genes are FXR1, HNRNPL, and YKT6.

Unless otherwise defined, all technical and scientific terms used herein have the same meaning as would be commonly understood by one of ordinary skill in the art to which the claimed invention belongs. Although methods and materials similar or equivalent to those described herein can be used to practice the methods and kits disclosed or claimed herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting. Other features and advantages of the claimed invention will be apparent from the following detailed description.

As used herein, the singular forms “a,” “an,” and “the” include plural referents unless the context clearly dictates otherwise. For example, reference to “a nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment disclosed or claimed herein.

As used herein, the terms “polynucleotide,” “nucleotide,” “oligonucleotide,” and “nucleic acid” can be used interchangeably to refer to nucleic acid comprising DNA, cDNA, RNA, derivatives thereof, or combinations thereof.

As used herein, the term “malignant” refers to a cancerous lesion. Malignant lesions can invade and destroy nearby tissue and metastasize to other parts of the body. Examples of pigmented malignant and pre-malignant lesions can include, but are not limited to melanoma, some basal cell and squamous cell carcinomas and actinic keratoses.

As used herein, the term “benign” refers to a lesion that is not cancerous. Benign lesions may grow larger but do not spread to other parts of the body. Also called non-malignant lesions. Examples of pigmented benign lesions can include, but are not limited to low-grade dysplastic nevi, congenital nevi and seborrheic keratoses.

As used herein, the term “intermediate risk” refers to a small subset (less than 5%, less than 4%, less than 3% or less than 2% of samples) of intermediate risk zone skin lesions, and these intermediate-risk zones may be evolving, borderline, or atypical and warrant special consideration in terms of patient management.

As used herein, the terms “metastasis” and “recurrence” are used interchangeably, and refer to the recurrence or disease progression that may occur locally (such as local recurrence and in transit disease), regionally (such as regional metastasis, nodal micrometastasis or macrometastasis), or distally (such as distal metastasis to brain, lung and/or other tissues). In certain embodiment, regional metastasis refers to a metastatic lesion within the regional nodal basin, including satellite or in-transit metastasis, but excluding local recurrence, and distant metastasis refers to metastasis beyond the regional lymph node basin. Risk, as used herein, includes low-risk, moderate-risk, or high-risk of metastasis according to any of the statistical methods disclosed herein.

In some embodiments, the methods described herein can comprise determining that the skin lesion is malignant or benign by combining with clinical staging factors recommended by, for example, the American Joint Committee on Cancer (AJCC), the Brigham Women's Hospital (BWH), the National Comprehensive Cancer Network (NCCN), the American Academy of Dermatology (AAD), or the American College of Mohs Surgeons (ACMS) to stage the skin lesion, or other histological features associated with risk of the skin lesion metastasis or disease-related death. In certain embodiments, the method further comprises identifying that the skin lesion is malignant or benign based on the probability score in combination with at least one risk factor, wherein the at least one risk factor is selected from skin lesion size, skin lesion location, immune status, perineural involvement (PNI), depth of invasion, differentiation, histological subtype, and lymphovascular invasion.

As used herein, the terms “skin lesion” or “suspicious skin lesion” or “pigmented skin lesion” refer to any tissue on or in the skin that has abnormal characteristics. For example, any lesions that are brown, black or blue in color, or may be confused with brown or black lesions. Pigmented skin lesions can comprise a mole, dark colored skin spot, or melanin containing skin area a pigmented skin lesion, such as a melanoma (malignant melanoma), a melanocytic nevus (mole), a basal cell carcinoma, and keratosis seborrheica, an angioma, or a hematoma.

A skin lesion sample may be obtained through a variety of sampling methods such as punch biopsy, shave biopsy, surgical excision (including Mohs micrographic surgery and wide local excision, or similar technique), core needle biopsy, incisional biopsy, endoscope ultrasound (EUS) guided-fine needle aspirate (FNA) biopsy, percutaneous biopsy, and other means of extracting RNA from the skin lesion of a patient. The most recognized risk factor for skin lesions is exposure to sunlight; thus, most pigmented skin lesions develop on sun-exposed skin sites, for example, the head or neck area. They can also be found on the face, ears, lips, trunk, arms, legs, hands, or feet. As used herein, the terms “risk factor” or “clinical staging factors” or “clinicopathologic factor” refer to any staging factor (i.e., risk factor) recommended by, for example, the American Joint Committee on Cancer (AJCC), the Brigham Women's Hospital (BWH), the National Comprehensive Cancer Network (NCCN), the American Academy of Dermatology (AAD), or the American College of Mohs Surgeons (ACMS), to stage a skin lesion, or other histological features associated with risk of skin lesions being malignant or benign. For example, a risk factors can include, but are not limited to size (any size on the head, neck, genitalia, hands, feet or pretibial surface (Areas H or M), or ≥2 cm size (or ≥1 cm if keratoacanthoma type) on any other area of the body (Area L)), location, immune status, perineural involvement (PNI; large (>0.1 mm), named nerve involvement, <0.1 mm in caliber, or unknown), depth of invasion (for example, any one or combination of: invasion beyond subcutaneous fat; depth ≥2 mm; and/or Clark level ≥IV), differentiation (i.e., poorly differentiated tumor histology), histological subtype (for example aggressive histological subtypes, which can be for example, any of acantholytic, adenosquamous, desmoplastic, sclerosing, basosquamous, small cell, spindle cell, infiltrating, clear cell, lymphoepithelial, sarcomatoid, or metaplastic subtypes), and lymphovascular invasion. Location definitions can be assigned according to the National Comprehensive Cancer Network (NCCN) Guidelines. For example, Area H, ‘mask areas’ of face (central face, eyelids, eyebrows, periorbital, nose, lips [cutaneous and vermillion], chin, mandible, preauricular and postauricular skin/sulci, temple, and ear), genitalia, hands, and feet; Area M, cheeks, forehead, scalp, neck, and pretibia; and Area L, trunk and extremities (excluding hands, nail units, pretibial, ankles, and feet). Immune status can refer to immunosuppressed, and types of immunosuppression can include patients that had an organ transplant, or have leukemia, lymphoma, or HIV.

The phrase “measuring the gene-expression levels” or “determining the gene-expression levels,” as used herein, refers to determining or quantifying RNA or proteins expressed by the gene or genes. The term “RNA” includes mRNA transcripts, and/or specific spliced variants of mRNA. The term “RNA product of the gene,” as used herein, refers to RNA transcripts transcribed from the gene and/or specific spliced variants. In some embodiments, mRNA is converted to cDNA before the gene expression levels are measured. With respect to proteins, gene expression refers to proteins translated from the RNA transcripts transcribed from the gene. The term “protein product of the gene” refers to proteins translated from RNA products of the gene. A number of methods can be used to detect or quantify the level of RNA products of the gene or genes within a sample, including microarrays, Real-Time PCR (RT-PCR; including quantitative RT-PCR), nuclease protection assays, RNA-sequencing (RNA-seq), and Northern blot analyses. In one embodiment, the assay uses the APPLIED BIOSYSTEMS™ HT7900 fast Real-Time PCR system. In addition, a person skilled in the art will appreciate that a number of methods can be used to determine the amount of a protein product of a gene of the methods disclosed herein, including immunoassays such as Western blots, ELISA, and immunoprecipitation followed by SDS-PAGE and immunocytochemistry. In certain embodiments, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR).

A person skilled in the art will appreciate that a number of detection agents can be used to determine gene expression. For example, to detect RNA products of the biomarkers, probes, primers, complementary nucleotide sequences, or nucleotide sequences that hybridize to the RNA products can be used. In another example, to detect cDNA products of the biomarkers, probes, primers, complementary nucleotide sequences, or nucleotide sequences that hybridize to the cDNA products can be used. To detect protein products of the biomarkers, ligands or antibodies that specifically bind to the protein products can be used.

As used herein, the term “hybridize” refers to the sequence specific non-covalent binding interaction with a complementary nucleic acid. In one embodiment, the hybridization is under high stringency conditions. Appropriate stringency conditions that promote hybridization are known to those skilled in the art.

As used herein, the terms “probe” and “primer” refer to a nucleic acid sequence that will hybridize to a nucleic acid target sequence. In one example, the probe and/or primer hybridizes to an RNA product of the gene or a complementary nucleic acid sequence. In another example, the probe and/or primer hybridizes to a cDNA product. The length of probe or primer depends on the hybridizing conditions and the sequences of the probe or primer and nucleic acid target sequence. In one embodiment, the probe or primer is at least 8, 10, 15, 20, 25, 50, 75, 100, 150, 200, 250, 400, 500, or more than 500 nucleotides in length. Probes and/or primers may include one or more label. Probes and/or primers may be commercially sourced from various providers (e.g., ThermoFisher Scientific). In certain embodiments, a label may be any substance capable of aiding a machine, detector, sensor, device, or enhanced or unenhanced human eye from differentiating a labeled composition from an unlabeled composition. Examples of labels include, but are not limited to: a radioactive isotope or chelate thereof, dye (fluorescent or non-fluorescent), stain, enzyme, or nonradioactive metal. Specific examples include, but are not limited to: fluorescein, biotin, digoxigenin, alkaline phosphates, biotin, streptavidin, ³H, ¹⁴C, ³²P, ³⁵S, or any other compound capable of emitting radiation, rhodamine, 4-(4′-dimethylamino-phenylazo)benzoic acid; 4-(4′-dimethylamino-phenylazo)sulfonic acid (sulfonyl chloride); 5-((2-aminoethyl)-amino)-naphtalene-1-sulfonic acid; Psoralene derivatives, haptens, cyanines, acridines, fluorescent rhodol derivatives, cholesterol derivatives; ethylene-diamine-tetra-acetic acid and derivatives thereof, or any other compound that may be differentially detected. The label may also include one or more fluorescent dyes. Examples of dyes include, but are not limited to: CAL-Fluor Red 610, CAL-Fluor Orange 560, dR110, 5-FAM, 6FAM, dR6G, JOE, HEX, VIC, TET, dTAMRA, TAMRA, NED, dROX, PET, BHQ+, Gold540, and LIZ.

As used herein, a “sequence detection system” is any computational method in the art that can be used to analyze the results of a PCR reaction. One example is the APPLIED BIOSYSTEMS™ HT7900 fast Real-Time PCR system. In certain embodiments, gene expression can be analyzed using, e.g., direct DNA expression in microarray, Sanger sequencing analysis, Northern blot, the NANOSTRING® technology, serial analysis of gene expression (SAGE), RNA-seq, tissue microarray, or protein expression with immunohistochemistry or western blot technique. PCR generally involves the mixing of a nucleic acid sample, two or more primers that are designed to recognize the template DNA, a DNA polymerase, which may be a thermostable DNA polymerase such as Taq or Pfu, and deoxyribose nucleoside triphosphates (dNTP's). Reverse transcription PCR, quantitative reverse transcription PCR, and quantitative real time reverse transcription PCR are other specific examples of PCR. In real-time PCR analysis, additional reagents, methods, optical detection systems, and devices known in the art are used that allow a measurement of the magnitude of fluorescence in proportion to concentration of amplified DNA. In such analyses, incorporation of fluorescent dye into the amplified strands may be detected or measured. In one embodiment, the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR).

As used herein, the terms “differentially expressed” or “differential expression” refer to a difference in the level of expression of the genes that can be assayed by measuring the level of expression of the products of the genes, such as the difference in level of messenger RNA transcript expressed (or converted cDNA) or proteins expressed of the genes. In one embodiment, the difference can be statistically significant. The term “difference in the level of expression” refers to an increase or decrease in the measurable expression level of a given gene as measured by the amount of messenger RNA transcript (or converted cDNA) and/or the amount of protein in a sample as compared with the measurable expression level of a given gene in a control, or control gene or genes in the same sample (for example, a non-recurrence sample). In another embodiment, the differential expression can be compared using the ratio of the level of expression of a given gene or genes as compared with the expression level of the given gene or genes of a control, wherein the ratio is not equal to 1.0. For example, an RNA, cDNA, or protein is differentially expressed if the ratio of the level of expression in a first sample as compared with a second sample is greater than or less than 1.0. For example, a ratio of greater than 1, 1.2, 1.5, 1.7, 2, 3, 3, 5, 10, 15, 20, or more than 20, or a ratio less than 1, 0.8, 0.6, 0.4, 0.2, 0.1, 0.05, 0.001, or less than 0.0001. In yet another embodiment, the differential expression is measured using p-value. For instance, when using p-value, a biomarker is identified as being differentially expressed as between a first sample and a second sample when the p-value is less than 0.1, less than 0.05, less than 0.01, less than 0.005, or less than 0.001.

The terms “increased expression” or “decreased expression,” as used herein, refer to an expression level of one or more genes, or prognostic RNA transcripts, or their corresponding cDNAs, or their expression products that has been found to be differentially expressed in malignant versus benign skin lesions. The higher the expression level of a gene that predominantly has increased expression in tumors of patients who had recurrence malignant skin lesion, the higher is the likelihood that the patient suffering from this skin lesion is expected to have a poor clinical outcome (i.e., higher risk of recurrence, metastasis, or both). In some embodiments, increased expression can be at least about 1.25-fold, at least about 1.5-fold, at least about 2-fold, at least about 3-fold, at least about 4-fold, at least about 5-fold, at least about 10-fold, at least about 20-fold, at least about 30-fold, at least about 40-fold, or at least about 50-fold increase in gene and/or protein levels compared to a control level or amount. In some embodiments, decreased expression can be at least about 1.25-fold, at least about 1.5-fold, at least about 2-fold, at least about 3-fold, at least about 4-fold, at least about 5-fold, at least about 10-fold, at least about 20-fold, at least about 30-fold, at least about 40-fold, or at least about 50-fold decrease in gene and/or protein levels compared to a control level or amount.

References herein to the “same” level of biomarker indicate that the level of biomarker measured in each sample is identical (i.e., when compared to the selected reference). References herein to a “similar” level of biomarker indicate that levels are not identical but the difference between them is not statistically significant (i.e., the levels have comparable quantities).

As used herein, the terms “control” and “standard” refer to a specific value that one can use to determine the value obtained from the sample. In one embodiment, a dataset may be obtained from samples from a group of subjects known to have a malignant skin lesion. The expression data of the genes in the dataset can be used to create a control (standard) value that is used in testing samples from new subjects. In such an embodiment, the “control” or “standard” is a predetermined value for each gene or set of genes obtained from subjects with a malignant skin lesion whose gene expression values and tumor types are known. In certain embodiments of the methods disclosed herein, non-limiting examples of control genes can include, but are not limited to, FXR1, HNRNPL, and YKT6. In some embodiments, a control population may comprise healthy individuals, individuals with cancer, or a mixed population of individuals with or without cancer. In certain embodiments, a control population may comprise individuals with non-metastatic cancer or cancer that did not recur.

As used herein, the term “normal” when used with respect to a sample population refers to an individual or group of individuals that does/do not have a particular disease or condition (e.g., malignant or benign) and is also not suspected of having or being at risk for developing the disease or condition. The term “normal” is also used herein to qualify a biological specimen or sample (e.g., a biological fluid) isolated from a normal or healthy individual or subject (or group of such subjects), for example, a “normal control sample.” The “normal” level of expression of a marker is the level of expression of the marker in cells in a similar environment or response situation, in a patient not afflicted with cancer. A normal level of expression of a marker may also refer to the level of expression of a “reference sample” (e.g., a sample from a healthy subject not having the marker associated disease). A reference sample expression may be comprised of an expression level of one or more markers from a reference database. Alternatively, a “normal” level of expression of a marker is the level of expression of the marker in non-tumor cells in a similar environment or response situation from the same patient that the tumor is derived from.

As used herein, the terms “gene-expression profile,” “GEP,” or “gene-expression profile signature” refer to any combination of genes, the measured messenger RNA transcript expression levels, cDNA levels, or direct DNA/RNA expression levels, or immunohistochemistry levels of which can be used to distinguish between two biologically different corporal tissues and/or cells and/or cellular changes. In certain embodiments, a gene-expression profile is comprised of the gene-expression levels of at least 12 discriminant genes of ABLMI1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1. In some embodiments, the gene set further comprises at least one control gene selected from FXR1, HNRNPL, and YKT6. In certain embodiments, the gene set further comprises 3 control genes or normalization genes that are FXR1, HNRNPL, and YKT6

In certain embodiments, a gene-expression profile is comprised of the gene-expression levels of at least 32, 31, 30, 29, 28, 27, 26, 25, 24, 23, 22, 21, 20, 19, 18, 17, 16, 15, 14, 13, or 12 discriminant genes, or fewer than 12 discriminant genes. In one embodiment, the gene-expression profile is comprised of 32 discriminant genes. In another embodiment, the gene-expression profile is comprised of 31 discriminant genes. In another embodiment, the gene-expression profile is comprised of 30 discriminant genes. In another embodiment, the gene-expression profile is comprised of 20 discriminant genes. In certain embodiments, the discriminant genes are selected from: ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1

In certain embodiments, there are 32 discriminant genes, and the 32 discriminant genes are: ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1. In some embodiments, the gene set further comprises 3 control genes or normalization genes that are: FXR1, HNRNPL, and YKT6.

As used herein, the term “predictive training set” refers to a cohort of skin lesions with known clinical outcome (i.e., malignant, intermediate risk or benign) and known genetic expression profile, used to define or establish all other skin lesions, based upon the genetic expression profile of each. Additionally, included in the predictive training set is the definition of “threshold points,” which are points at which a classification of metastatic risk is determined, specific to each individual gene expression level.

As used herein, the term “altered in a predictive manner” refers to changes in genetic expression profile that identifies or determines a skin lesion to be malignant, intermediate risk or benign. Predictive modeling can be measured as: 1) identifies or determines a skin lesion to be malignant, intermediate risk or benign; and/or 2) a linear outcome based upon a probability score from 0 to 1 that reflects the correlation of the genetic expression profile of a skin lesion with the genetic expression profile of the samples that comprise the training set used to identifies or determines a skin lesion to be malignant, intermediate risk or benign. The increasing probability score from 0 to 1 reflects incrementally increasing accuracy of a malignant lesion. Within the probability score range from 0 to 1, a probability score, for example, of less than 0.5 reflects a sample with a low risk of a malignant lesion, while a probability score, for example, of greater than 0.5 reflects a sample with a high risk of a malignant lesion.

The TNM (Tumor-Node-Metastasis) status system is the most widely used cancer staging system among clinicians and is maintained by the American Joint Committee on Cancer (AJCC) and the International Union for Cancer Control (UICC). Cancer staging systems codify the extent of cancer to provide clinicians and patients with the means to quantify prognosis for individual patients and to compare groups of patients in clinical trials and who receive standard care around the world.

As used herein, the terms “treatment,” “treat,” or “treating” refer to a method of reducing the effects of a disease or condition or symptom of the disease or condition. Thus, in the methods disclosed herein, treatment can refer to a 5%, 10%, 20%, 30%, 40%, 50%, 60%, 70%, 80%, 90%, or 100% reduction in the severity of an established disease or condition or symptom of the disease or condition. For example, a method of treating a disease is considered to be a treatment if there is a 5% reduction in one or more symptoms of the disease in a subject as compared to a control. Thus, the reduction can be a 5%, 10%, 20%, 30%, 40%, 50%, 60%, 70%, 80%, 90%, 100% or any percent reduction between 5% and 100% as compared to native or control levels. It is understood that treatment does not necessarily refer to a cure or complete ablation of the disease, condition, or symptoms of the disease or condition. After a skin lesion is found and determined to be malignant, intermediate risk, or benign, a medical professional or team of medical professionals will recommend one or several treatment options. In determining a treatment plan, factors to consider include the type, location, and stage of the cancer, as well as the patient's overall physical health. Patients with suspicious skin lesions typically are managed by a health care team made up of doctors from different specialties, such as: a dermatologist (in particular, a dermatologist who specializes in Mohs micrographic surgery), an orthopedic surgeon (in particular, a surgeon who specializes in diseases of the bones, muscles, and joints), a surgical oncologist, a thoracic surgeon, a medical oncologist, a radiation oncologist, and/or a physiatrist (or rehabilitation doctor). After a skin lesion is found and determined to be malignant, intermediate risk, or benign, a medical professional or team of medical professionals will typically recommend one or several treatment options including one or more of surgery, radiation, chemotherapy, and targeted therapy.

The NCCN Guidelines® define low risk tumors as tumors that involve: (1) an area of less than 20 mm (for truck and extremities) or less than 10 mm for the cheeks, forehead, scalp, neck and pretibial; (2) well defined borders; (3) primary tumor; (4) not rapidly growing; (5) from a patient who has no neurologic symptoms and is not considered immunosuppressed; (6) from a site free of chronic inflammation; (7) well or moderately differentiated; (8) free of acantholytic, adenosquamous, desmoplastic, or metaplastic subtypes; (9) depths of less than 2 mm; and (10) free of perineural, lymphatic, or vascular involvement.

The NCCN Guidelines® define high risk tumors as tumors that involve: (1) an area of greater than 20 mm (for trunk and extremities), greater than 10 mm for the cheeks, forehead, scalp, neck and pretibial, or any involving the “mask areas” (such as central face, eyelids, eyebrows, periorbital, nose, lips, chin, mandible, temple or ear), genitalia, hands and feet; (2) poorly defined borders; (3) recurrent tumor; (4) rapidly growing; (5) from a patient who has neurologic symptoms or is considered immunosuppressed; (6) from a site with chronic inflammation; (7) poorly differentiated; (8) presence of acantholytic, adenosquamous, desmoplastic, or metaplastic subtypes; (9) depths of greater than or equal 2 mm; and (10) presence of perineural, lymphatic, or vascular involvement.

As used herein, the term “aggressive cancer treatment regimen” refers to a treatment regimen that is determined by a medical professional or team of medical professionals and can be specific to each patient. In certain embodiments, a skin lesion determined to be malignant using the methods and kits disclosed herein, would be treated using an aggressive cancer treatment regimen. For patients diagnosed with a malignant skin lesion, or having a high likelihood of having melanoma, aggressive treatment is preferred. Those skilled in the art are familiar with various other aggressive and less aggressive treatments for each type of cancer. For patients with a low likelihood of a malignant skin lesion, a less aggressive therapy could be prescribed. Therefore, for a patient having a low risk of having a malignant skin lesion or melanoma, a medical provider could recommend a regime of “watchful-waiting.” If a patient has an indeterminate likelihood a malignant skin lesion or melanoma, further testing could be prescribed. Therefore, for a patient having an indeterminate score, a medical provider could recommend further testing of the skin lesion. Whether a treatment is considered to be aggressive will generally depend on the cancer-type, the age of the patient, and other factors known to those of skill in the art. For example, in breast cancer, adjuvant chemotherapy is a common aggressive treatment given to complement the less aggressive standards of surgery and hormonal therapy. Those skilled in the art are familiar with various other aggressive and less aggressive treatments for each type of cancer. An aggressive cancer treatment regimen is defined by the National Comprehensive Cancer Network (NCCN), and has been defined in the NCCN Guidelines® as including one or more of: 1) imaging (CT scan, PET/CT, MM, chest X-ray), 2) discussion and/or offering of tumor resection if a tumor is determined to be resectable (e.g., by Mohs micrographic surgery or resection with complete circumferential margin assessment), 3) radiation therapy (RT), 4) chemoradiation, 5) chemotherapy, 6) regional limb therapy, 7) palliative surgery, 8) systemic therapy, 9) immunotherapy, and 10) inclusion in ongoing clinical trials. Guidelines for clinical practice are published in the National Comprehensive Cancer Network (NCCN Guidelines® Squamous Cell Skin Cancer Version 2.2018, updated Oct. 5, 2017, available on the World Wide Web at NCCN.org).

Additional therapeutic options may include, but are not limited to: 1) combination regimens such as: AD (doxorubicin, dacarbazine); AIM (doxorubicin, ifosfamide, mesna); MAID (mesna, doxorubicin, ifosfamide, dacarbazine); ifosfamide, epirubicin, mesna; gemcitabine and docetaxel; gemcitabine and vinorelbine; gemcitabine and dacarbazine; doxorubicin and olaratumab; methotrexate and vinblastine; tamoxifen and sulindac; vincristine, dactinomycin, cylclophosphamide; vincristine, doxorubicin, cyclophosphamide; vincristine, doxorubicin, cyclophosphamide with ifosfamide and etoposide; vincristine, doxorubicin, ifosfamide; cyclophosphamide topotecan; or ifosfamide, doxorubicin; and/or 2) single agents, such as: cisplatin or other metallic compounds, 5-FU/capecitabine (Xeloda®), cetuximab (Erbitux®), cemiplimab (Libtayo®), pembrolizumab (MK-3475), panitumumab (Vectibix®), dacomitinib (PF-00299804), gefitinib (ZD1839, Iressa), doxorubicin, ifosfamide, epirubicin, gemcitabine, dacarbazine, temozolomide, vinorelbine, eribulin, trabectedin, pazopanib, imatinib, sunitinib, regorafenib, sorafenib, nilotinib, dasatinib, interferon, toremifene, methotrexate, irinotecan, topotecan, paclitaxel, nab-paclitaxel (abraxane), docetaxel, bevacizumab, temozolomide, sirolimus (Rapamune®), everolimus, temsirolimus, crizotinib, ceritinib, or palbociclib.

While surgical excision remains the mainstay for treating operable (Stage I-III) malignant skin lesions, for Stage I patients, en bloc resection with negative margins is generally considered sufficient for long-term local control. For those with incomplete excision margins and/or other unfavorable pathologic features, pre- or post-operative chemotherapy and/or radiation treatment can be recommended. No therapy has shown consistent efficacy for the treatment of excised skin lesions, and treatment options for unresectable or advanced malignant skin lesions can be limited.

Immunotherapy using an anti-PD1 inhibitor has shown promising results in early phase studies with melanoma patients. Examples of immunotherapies (that can be used alone or in combination with any one or more of tumor resection if a tumor is determined to be resectable, radiation therapy, chemoradiation, chemotherapy, regional limb therapy, palliative surgery, systemic therapy, additional immunotherapeutic, or inclusion in ongoing clinical trials), can include, for example, pembrolizumab (Keytruda®) and nivolumab (Opdivo®), cemiplimab (Libtayo®; a fully human monoclonal antibody to Programmed Death-1). PD-1 is a protein on T-cells that normally help keep T-cells from attacking other cells in the body. By blocking PD-1, these drugs can boost the immune response against cancer cells. CTLA-4 inhibitors (for example, ipilimumab (Yervoy®)) are another class of drugs that can boost the immune response. In some instances, cytokine therapy (such as, interferon-alpha and interleukin-2) can be used to boost the immune system. Examples of interferon and interleukin-based treatments can include, but are not limited to, aldesleukin (Proleukin®), interferon alpha-2b (INTRON®), and pegylated interferon alpha-2b (Sylvatron®; PEG-INTRON®, PEGASYS). In another embodiment, oncolytic virus therapy can be used. Along with killing the cells directly, the oncolytic viruses can also alert the immune system to attack the cancer cells. For example, talimogene laherparepvec (Imlygic®), also known as T-VEC, is an oncolytic virus that can be used to treat melanomas. Additional immunotherapies may include CV8102.

Additionally, targeted therapies may be used to treat patients with malignant skin lesions. For example, targeted therapies can include, but are not limited to, vemurafenib (Zelboraf®), dabrafenib (Tafinlar®), trametinib (Mekinist®), CLL442, and cobimetinib (Cotellic®). These drugs target common genetic mutations, such as the BRAFV600 mutation, that may be found in a subset of patients.

In certain embodiments, the methods as disclosed herein can be used to determine a recommended risk-aligned management plan. For example, patients determined to have a low risk skin lesion (benign) can be managed under a low intensity management plan. A low intensity management plan can comprise minimal clinical follow-up (e.g., 1-2× per year), a reduced imaging (low frequency or no imaging performed), a reduced nodal assessment (palpation only), and/or an avoidance of adjuvant radiation or chemotherapy. For example, patients determined to have an intermediate risk skin lesion can be managed under a moderate intensity management plan. A moderate intensity management plan can comprise a high frequency of clinical follow-up (e.g., 2-4× per year for about 3 years), imaging (e.g., baseline and annual nodal US/CT for 2 years), consideration of nodal biopsy or elective neck dissection, and/or a consideration of adjuvant radiation or chemotherapy. For example, patients determined to have a malignant skin lesion can be managed under a high intensity management plan. A high intensity management plan can comprise the highest frequency of clinical follow-up (e.g., 4-12× per year for about 3 years), imaging (e.g., baseline and 4× per year nodal US/CT for 2 years), recommendation of nodal biopsy or elective neck dissection, and/or a recommendation of adjuvant radiation, chemotherapy, and/or clinical trials. Importantly, these risk-stratified management plans fall within the current NCCN Guidelines® for patients identified as having a tumor as defined by clinical and pathologic features only.

As used herein, the term “adjuvant therapy” refers to additional cancer treatment given after a primary treatment to lower the risk that the cancer will recur. For example, adjuvant therapy is often used before and/or after a primary surgical treatment in order to decrease the chance of the primary cancer recurring. In surgery, where all detectable disease has been removed, there remains a statistical risk of relapse or recurrence due to the presence of undetected disease. Adjuvant therapy given before the primary treatment is called neoadjuvant therapy. Neoadjuvant therapy can also decrease the chance of the cancer recurring, and it's often used to make the primary treatment, such as an operation or radiation treatment more effective. Adjuvant therapy can include chemotherapy, radiation therapy, hormone therapy, targeted therapy, immunotherapies, or biological therapy.

In some embodiments, the skin lesion is a frozen sample. In another embodiment, the skin lesion sample is formalin-fixed and paraffin embedded. In certain embodiments, the skin lesion sample is taken from a formalin-fixed, paraffin embedded wide local excision sample. In another embodiment, the skin lesion tumor is taken from a formalin-fixed, paraffin embedded primary biopsy sample. In some embodiments, the skin lesion sample can be from image guided surgical biopsy, shave biopsy, wide excision, or a lymph node dissection.

In certain embodiments, analysis of genetic expression and determination of outcome is carried out using radial basis machine and/or partial least squares analysis (PLS), partition tree analysis, logistic regression analysis (LRA), K-nearest neighbor, neural networks, ensemble learners, voting algorithms, or other algorithmic approach. These analysis techniques take into account the large number of samples required to generate a training set that will enable accurate prediction of outcomes as a result of cut-points established with an in-process training set or cut-points defined for non-algorithmic analysis, but that any number of linear and nonlinear approaches can produce a statistically significant and clinically significant result. As used herein, the term “Kaplan-Meier survival analysis” is understood in the art to be also known as the product limit estimator, which is used to estimate the survival function from lifetime data. In medical research, it is often used to measure the fraction of patients living for a certain amount of time after treatment. JMP GENOMICS®, R, Python libraries including SciPy, SciKit, and NumPy software or systems such as TensorFlow provides an interface for utilizing each of the predictive modeling methods disclosed herein, and should not limit the claims to methods performed only with JMP GENOMICS®, R, Python, or TensorFlow software.

Kits can include any combination of components that facilitates the performance of an assay. A kit that facilitates assessing the expression of the gene or genes may include suitable nucleic acid-based and/or immunological reagents as well as suitable buffers, control reagents, and printed protocols. A “kit” is any article of manufacture (e.g., a package or container) comprising at least one reagent, e.g., a probe, oligo, or primer set, for specifically detecting a marker or set of markers used in the methods disclosed herein. In some embodiments, a kit comprises one more probes capable of selectively hybridizing to at least one of the genes in Table 3. The terms “probe” and “oligonucleotide” (also “oligo”), when used in the context of nucleic acids, interchangeably refer to a relatively short nucleic acid fragment or sequence. The invention also provides primers useful in the methods of the invention. “Primers” are probes capable, under the right conditions and with the right companion reagents, of selectively amplifying a target nucleic acid (e.g., a target gene). In the context of nucleic acids, “probe” is used herein to encompass “primer” since primers can generally also serve as probes. The article of manufacture may be promoted, distributed, sold, or offered for sale as a unit for performing the methods disclosed herein. The reagents included in such a kit comprise probes, primers, or antibodies for use in detecting one or more of the genes and/or gene sets disclosed herein and demonstrated to be useful for determining a malignant or benign skin lesion, in patients with a suspicious skin lesion. Kits that facilitate nucleic acid based methods may further include one or more of the following: specific nucleic acids such as oligonucleotides, labeling reagents, enzymes including PCR amplification reagents such as Taq or Pfu, reverse transcriptase, or other, and/or reagents that facilitate hybridization. In addition, the kits disclosed herein may preferably contain instructions which describe a suitable detection assay. Such kits can be conveniently used, e.g., in clinical settings, to diagnose and evaluate patients exhibiting symptoms of cancer, in particular patients exhibiting the possible presence of a malignant skin lesion.

### EXAMPLES

The Examples that follow are illustrative of specific embodiments of the claimed invention, and various uses thereof. They are set forth for explanatory purposes only, and should not be construed as limiting the scope of the claimed invention in any way.

### Example 1: Sample Preparation and Expression Analysis

Sample and Clinical Data Collection

Archival benign samples and associated de-identified clinical data were collected from multiple independent dermatopathology laboratories as part of this Institutional Review Board (IRB)-approved study. Formalin-fixed, paraffin-embedded (FFPE) pigmented lesion tissue was collected as 5 μm sections for subsequent diagnosis based on H&E staining and for real-time quantitative reverse transcription PCR (qRT-PCR) analysis. Additionally, archival melanoma samples and de-identified clinical data were obtained from specimens submitted to Castle Biosciences for clinical testing with the 31-GEP (DecisionDx®-Melanoma). A total of 951 samples diagnosed between January 2013 and August 2020 were included in the training and validation cohorts, of which 498 were benign and 453 malignant. All laboratory personnel were blinded to clinical diagnoses for all 951 samples.

Samples were excluded from the study if there was less than 10% tumor volume (cellularity of all samples was determined by a single dermatopathologist), tissue originated from melanoma metastases, lesions were not primary to the skin, tissue was derived from re-excisions (including wide local excision), diagnosis was a non-melanocytic neoplasm, and if patients had previous radiation or immunotherapy treatment. Melanoma subtypes of acral lentiginous, desmoplastic, lentiginous, lentigo maligna, nevoid, nodular, spitzoid, superficial spreading, and melanoma in situ were included. Benign subtypes of blue nevus, common nevus (compound, junctional and intradermal), deep penetrating nevus, dysplastic nevus (compound and junctional), and Spitz nevus were included.

Histopathologic Examination

Eight dermatopathologists participated in sample acquisition, and six dermatopathologists participated in sample review for diagnostic concordance. The majority of these dermatopathologists are affiliated with private practice and have an average of 12 years of experience reviewing skin lesions. All acquired samples were received with the original pathology report. For all benign diagnoses, the contributing dermatopathologists provided a description of the lesion in a free text field, and the information was entered into the clinical research form. All benign samples then underwent H&E diagnostic review by a second and third dermatopathologist who were blinded to the original diagnosis and provided with only patient age and anatomic location of the lesion. Reviewing dermatopathologists were asked to select a diagnosis (benign, malignant, or unknown (unknown malignant potential (UMP)) as well as a subtype classification from a pre-determined list. If discordance was observed across three diagnoses, the case was reviewed by additional dermatopathologists in a blinded manner for adjudication. A total of 405 samples that were diagnosed as benign by 3 out of 3 dermatopathologists were included in the study, additionally, 78 cases diagnosed as UMP by no more than 1 dermatopathologist (i.e. 2 benign and 1 UMP) were added to the training and validation cohorts. As a result, the final training and validation cohorts consisted of benign samples with full diagnostic concordance (167/200 and 228/273 of samples, respectively) and samples with no more than one UMP classification (33/200 and 45/273, respectively).

Real-Time Quantitative Reverse-Transcription PCR

Pigmented lesions were processed for qRT-PCR expression analysis in a central CLIA-certified, CAP-accredited, and New York State Department of Health permitted laboratory. Tumor sections were macrodissected from unstained FFPE tissue and total RNA was extracted per manufacturer's instructions using either the QlAsymphony SP Automated Nucleic Acid Extractor (Qiagen) or KingFisher Flex (ThermoFisher Scientific) platforms. Total RNA concentration was quantified using the NanoDrop 8000 (ThermoFisher Scientific). cDNA was obtained using the High-Capacity cDNA Reverse Transcription Kit (Applied Biosystems). cDNA pre-amplification reaction was performed utilizing the TaqMan PreAmp Master Mix (Applied Biosystems) and a 14-cycle amplification. Pre-amplified samples were diluted 1:2.5× in TE Buffer pH 7.0 (ThermoFisher Scientific) and combined with an equal volume of 2× Open Array Real-Time PCR Master Mix (Applied Biosystems). The samples were loaded onto a custom Open Array gene card using the QuantStudio 12K Flex AccuFill system (Applied Biosystems) subsequently run on the QuantStudio 12K PCR system.

Expression Analysis and Diagnosis Assignment

The array data was analyzed to identify genes that were best able to segregate benign and malignant lesions based on levels of gene expression. The resulting gene set was then reviewed to ensure that a wide variety of biological pathways were represented and to confirm the biological relevance of those genes. As a result, 76 candidate diagnostic genes were selected for model training. Three genes (FXR1, HNRNPL, and YKT6) were reliably and consistently expressed in the Study Cohort and chosen as control genes. Triplicate gene expression data were aggregated and normalized using control probes. Failure of three or more candidate genes (MGF, multiple gene failure) led to sample exclusion from training and validation cohorts, while control genes were evaluated independently, and failure of any control gene resulted in sample exclusion. Following quality control measures to assess amplification and stability of gene expression, 58 discriminant probes and 3 control probes were selected for further analysis. Deep learning techniques were applied to gene expression data for gene selection and model identification. Gene expression data analyzed with neural network modeling resulted in two diagnostic algorithms. Tumors with spitzoid or melanoma in situ features had poorer initial classification accuracy; therefore, the presence of those features in diagnosis was added to the input of the algorithm to improve accuracy. Algorithm improvement continued until the mean kappa value improved by less than 0.01 for the top 25% of the assay population. Hyperparameter selection and model evaluation was performed using 4×4-fold cross validation. Kappa was determined from the average kappa value at each of the cross validation runs. The final model was trained against all training data using the optimal gene set. Two models were developed which together generated the locked algorithm for the 35-GEP test. Classification into benign (gene expression profile suggestive of benign neoplasm), intermediate-risk (gene expression profile cannot exclude malignancy) or malignant (gene expression profile suggestive of malignant melanoma) zones was determined from the probability scores from both algorithms.

Analysis was performed with R v.3.3.3. Differences in age were assessed using the Wilcoxon F test. Differences in categorical variables including sex, ulceration status and location were assessed by Pearson Chi-square test. P values <0.05 were considered statistically significant.

Sample Cohorts

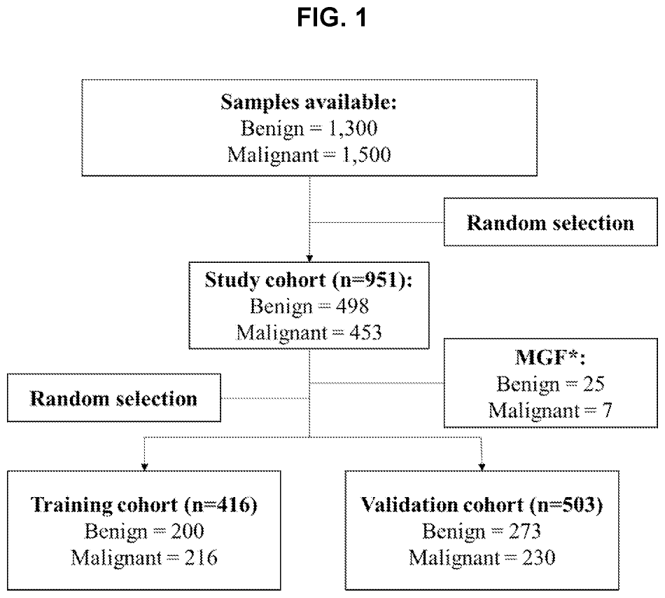

Quantitative RT-PCR was performed on 498 benign and 453 malignant lesions accrued under an IRB-approved protocol in a multicenter cohort (FIG. 1). Thirty-two samples (˜3.4%) of the Study Cohort were excluded from further analysis due to MGF with the remaining 919 samples randomized to training or validation cohorts while conserving benign or melanoma subtype representation in each cohort. Training (200 benign nevi and 216 melanomas) and validation (273 benign nevi and 230 melanomas) cohorts' demographic details are shown in Table 1.

TABLE 1
NevDemographic information for training and validation cohorts.

| | Training cohort^{†} | | | | Validation cohort^{†} | | | |
|---|---|---|---|---|---|---|---|---|
| | Melanoma | | Benign nevi | | Melanoma | | Benign nevi | |
| | N = 216 | | N = 200 | | N = 230 | | N = 273 | |
| Age, median (range) | 66 | (18-93) | 47 | (7-85) | 67 | (25-98) | 48 | (2-90) |
| Sex, % male | 55 | | 46 | | 63 | | 39 | |
| Breslow thickness, mm (range) | 1.22 | (0-10) | NA | | 1.23 | (0.1-4.9) | NA | |
| T stage, % (n) | | | | | | | | |
| T1a | 29 | (56) | — | | 23 | (48) | — | |
| T1b | 13 | (25) | — | | 20 | (42) | — | |
| T2a | 16 | (31) | — | | 16.5 | (35) | — | |
| T2b | 14 | (27) | — | | 11 | (23) | — | |
| T3a | 11.5 | (23) | — | | 17.5 | (37) | — | |
| T3b | 16 | (31) | — | | 11 | (23) | — | |
| T4b | 0.5 | (1) | — | | 1 | (2) | — | |
| Ulceration % (n) | | | | | | | | |
| Present | 29.5 | (64) | — | | 23.5 | (54) | — | |
| Absent | 70.5 | (152) | — | | 76.5 | (176) | — | |
| Not addressed | — | | 100 | (200) | — | | 100 | (273) |
| Location on body, % (n) | | | | | | | | |
| Abdomen/Chest | 8 | (18) | 11 | (22) | 5.5 | (13) | 11.5 | (32) |
| Acral | 3 | (6) | 1 | (2) | 2 | (5) | 1 | (2) |
| Back | 27 | (58) | 36.5 | (73) | 29 | (67) | 41 | (113) |
| Extremities | 40 | (86) | 23 | (46) | 40 | (91) | 20 | (54) |
| Head/neck | 20 | (43) | 24 | (48) | 22 | (50) | 23 | (63) |
| Other | 2 | (5) | 4.5 | (9) | 1.5 | (4) | 3.5 | (9) |

^{†}No statistically significant differences were observed in the training vs. validation cohorts.
NA—not addressed.

No statistically significant differences were observed in the training vs. validation cohorts. The median age of patients with benign lesions was 47 (range 7-85) years in the training cohort and 48 (2-90) years in the validation cohort (p=0.944), while patients with malignant tumors had a median age of 66 (range 18-93) and 67 (25-98) years of age (p=0.203), respectively. Training and validation cohorts had 55% and 63% (p=0.071) male patients with malignant diagnosis, while 46% and 39% (p=0.17) males were included in training and validation, respectively. Ulceration was present in 29.5% (64/216) melanomas in training and 23.5% (54/230) melanomas in validation cohorts (p=0.141). The majority of malignant lesions were biopsied from arms and legs (extremities, 40% of cases in training and validation, p=0.812), while benign lesions were mainly located on patients' backs (36.5% in training cohort and 41% in validation, p=0.863). The distribution of different subtypes of melanoma and nevi in the training and validation sets are provided in Table 2.

TABLE 2
Distribution of subtypes in training and validation cohorts.

| | Training cohort, n | Validation cohort, n | P value |
|---|---|---|---|
| Melanoma | 216 | 230 | 0.399 |
| Acral lentiginous | 6 | 5 | |
| Desmoplastic | 15 | 14 | |
| Lentiginous | 3 | 3 | |
| Lentigo maligna | 23 | 26 | |
| In situ | 21 | 19 | |
| Nevoid | 15 | 15 | |
| Nodular | 47 | 60 | |
| Superficial spreading | 66 | 77 | |
| Spitzoid | 15 | 3 | |
| Not specified | 5 | 8 | |
| Nevi | 200 | 273 | 0.468 |
| Blue | 38 | 45 | |
| Common nevi | | | |
| Compound | 16 | 16 | |
| Intradermal | 20 | 41 | |
| Junctional | 10 | 10 | |
| Not specified | 30 | 32 | |
| Deep penetrating | 1 | 2 | |
| Dysplastic | | | |
| Compound | 40^{a} | 49^{b} | |
| Junctional | 28^{c} | 42^{d} | |
| Spitz | 17 | 36 | |

P value was calculated using the Pearson Chi-square test.
Dysplastic nevi had different degrees of atypia: ^{a}mild (n = 19), moderate (n = 4) and severe (n = 3); ^{b}mild (n = 24), moderate (n = 2), and severe (n = 3); ^{c}mild (n = 20) and moderate (n = 6); ^{d}mild (n = 22) and moderate (n = 17) atypia.

### Example 2: Development of 35-GEP Profile

Artificial neural networks were selected as the model type due to their ability to recognize multiple patterns, which is crucial to successfully distinguish large number of different subtypes of benign nevi and melanomas. Therefore, to represent biological diversity and different growth patterns and features, lesions unanimously diagnosed as benign or malignant by 3/3 reviewers and lesions with less definitive histopathology resulting in 2/3 concordance were included in the training set to ensure the resulting algorithm is capable of classifying both typical and heterogenous lesions. A 35-GEP comprising 32 discriminant genes and 3 control genes was developed using neural networks for model fitting and genetic algorithms for feature selection on a diverse set of benign and malignant samples. The 35-GEP is primarily composed of genes in cytoskeletal and barrier functions, gene regulation and melanin biosynthesis (Table 3). Multiple molecular pathways have been associated with melanoma progression and the 35-GEP signature includes several genes from key signaling networks to encompass the complexity of the disease. Biological processes such as epithelial cell differentiation, tissue and epidermis development, programmed cell death, and keratinocyte differentiation were identified as top functional enrichments for the gene set.

TABLE 3
Genes included in the 35-GEP and their functions.

| Gene classification | Gene symbol | Gene name |
|---|---|---|
| Barrier function | HAL | Histidine ammonia-lyase |
| Barrier function | MGP* | Matrix Gla protein |
| Barrier function | CST6* | Cystatin-M |
| Barrier function | GJA1* | Gap junction alpha-1 protein |
| Barrier function | CSTA | Cystatin A |
| Barrier function | CLCA2* | Calcium-activated chloride |
| | | channel regulator 2 |
| Cytoskeleton involved | KRT17 | Keratin, type I cytoskeletal 17 |
| Cytoskeleton involved | PPL* | Periplakin |
| Cytoskeleton involved | KRT2 | Keratin 2 |
| Cytoskeleton involved | ABLIM1 | Actin binding LIM protein 1 |
| Cytoskeleton involved | DSP | Desmoplakin |
| Cytoskeleton involved | NES | Nestin |
| Gene regulation | KLF5 | Kruppel-like factor 5 |
| Gene regulation | GATA3 | GATA binding protein 3 |
| Gene regulation | BAP1* | Ubiquitin carboxyl-terminal |
| | | hydrolase BAP1 |
| Gene regulation | TP63 | Tumor Protein P63 |
| Gene regulation | SAP130* | Histone deacetylase complex |
| | | subunit SAP130 |
| Gene regulation | SFN | 14-3-3 protein sigma |
| Melanin Biosynthesis | GPR143 | G-protein coupled receptor 143 |
| Melanin Biosynthesis | WIPI1 | WD repeat domain |
| | | phosphoinositide-interacting |
| | | protein 1 |
| Melanin Biosynthesis | DCT | Dopachrome tautomerase |
| Melanin Biosynthesis | ATP6V0E2 | ATPase H+ transporting |
| | | V0 subunit E2 |
| Melanin Biosynthesis | PTN | Pleiotrophin |
| Protein synthesis | RPS16 | 40S ribosomal protein S16 |
| Protein synthesis | RPL37A | 60S ribosomal protein L37a |
| Tumorigenesis | BCL2A1 | Bcl-2-related protein A1 |
| Tumorigenesis | BTG1* | Protein BTG1 |
| Tumorigenesis | ANXA8L1 | Annexin A8-like protein 1 |
| Tumorigenesis | DUSP4 | Dual specificity protein |
| | | phosphatase 4 |
| Tumorigenesis | CXCL14* | C-X-C motif chemokine 14 |
| Tumorigenesis | S100A8* | Protein S100-A8 |
| Tumorigenesis | S100A9* | Protein S100-A9 |
| Housekeeping | FXR1 | RNA binding protein |
| Housekeeping | HNRNPL | mRNA function protein |
| Housekeeping | YKT6 | ER membrane protein |

*Eleven discriminant genes are also included in the 31-GEP test.

### Example 3: Validation of the 35-GEP

Accuracy metrics within the validation cohort (all ages included) were 99.1% (95% CI: 97.9-100%) sensitivity, 94.3% (95% CI: 91.5-97.1%) specificity, 93.6% (95% CI: 90.5-96.7%) positive predictive value (PPV) and 99.2% (95% CI: 98.1-100%) negative predictive value (NPV) (Table 4), suggesting that the 35-GEP test could be a highly accurate ancillary diagnostic test for diagnosis of melanocytic neoplasms. In a patients >18 years old the 35-GEP had sensitivity of 99.1% (95% CI: 97.9-100%), specificity of 96.2% (95% CI: 93.8-98.6%), PPV of 96.1% (95% CI: 93.6-98.6%) and NPV of 99.1% (95% CI: 97.9-100%) (Table 4). Accuracy metrics were calculated without the inclusion of lesions identified as intermediate-risk (3.6% and 3.8% of the total samples in all ages and >18 years old, respectively). Overall, the 35-GEP was able to accurately classify different subtypes of melanoma and nevi as benign or malignant (Table 5). The 35-GEP accurately classified melanoma lesions as malignant in 14/14 desmoplastic melanomas, 25/26 lentigo maligna, 15/15 nevoid, 59/60 nodular, 72/77 superficial spreading, and 17/19 melanoma in situ. Furthermore, nevi were also appropriately classified as benign for 42/45 blue, 96/99 common nevi (including 15 compound, 40 intradermal and 10 junctional), 82/91 dysplastic nevi (including 44 compound and 38 junctional), and 26/36 Spitz nevi received a benign diagnosis.

TABLE 4
the 35-GEP accuracy metrics.

| | All ages | | >18 years old | |
|---|---|---|---|---|
| | N = 503 | | N = 478 | |
| | 35-GEP | 95% CI | 35-GEP | 95% CI |
| Sensitivity | 99.1% | 97.9-100 | 99.1% | 97.9-100 |
| Specificity | 94.3% | 91.5-97.1 | 96.2% | 93.8-98.6 |
| PPV | 93.6% | 90.5-96.7 | 96.1% | 93.6-98.6 |
| NPV | 99.2% | 98.1-100 | 99.1% | 97.9-100 |

| | | |
|---|---|---|
| Intermediate-risk | 3.6% | 3.8% |
| result | | |

Samples that fall in intermediate-risk zone were excluded from the calculation.
PPV—positive predictive value; NPV—negative predictive value; CI—confidence interval.

TABLE 5
Performance of the 35-GEP in different
subtypes of nevi and melanoma.

| | 35-GEP result | | |
|---|---|---|---|
| | Benign, n | Intermediate-risk, n | Malignant, n |
| Melanomas | 2 | 8 | 220 |
| Acral lentiginous | | | 5 |
| Desmoplastic | | | 14 |
| Lentiginous | | | 3 |
| Lentigo maligna | | 1 | 25 |
| In situ | 1 | 1 | 17 |
| Nevoid | | | 15 |
| Nodular | 1 | | 59 |
| Spitzoid | | 1 | 2 |
| Superficial spreading | | 5 | 72 |
| Not specified | | | 8 |
| Nevi | 248 | 10 | 15 |
| Blue | 42 | 2 | 1 |
| Common nevi | | | |
| Compound | 15 | | 1 |
| Intradermal | 40 | | 1 |
| Junctional | 10 | | |
| Not specified | 31 | | 1 |
| Deep penetrating nevus | 2 | | |
| Dysplastic | | | |
| Compound | 44^{a} | 4^{b} | 1^{c} |
| Junctional | 38^{d} | 1^{e} | 3^{f} |
| Spitz | 26 | 3 | 7 |

Dysplastic nevi had different degrees of atypia: ^{a}mild (n = 22), moderate (n = 2) and severe (n = 3); ^{b}mild (n = 1); ^{c}mild (n = 1); ^{d}mild (n = 21) and moderate (n = 14); ^{e}moderate (n = 1); ^{f}mild (n = 1) and moderate (n = 2) atypia

Out of 230 melanomas, two were identified as benign, while 15 out of 273 benign lesions were classified as malignant (Table 6). The nodular melanoma with the Breslow thickness of 4.0 mm had the low-risk prognostic Class 1B 31-GEP result. Among the 15 benign lesions that were classified as malignant by the 35-GEP, four were dysplastic (one compound with mild atypia and three junctional with mild/moderate atypia), one compound nevi, one combined blue and intradermal nevus, one blue nevus, one benign melanocytic nevus (not otherwise specified), and seven were Spitz nevi. Six of the seven misclassified Spitz nevi were in pediatric patients suggesting this may be a limitation of the 35-GEP. Spitzoid lesions are particularly difficult to diagnose as many have ambiguous histologic characteristics and may involve regional lymph nodes in the absence of increased mortality rates or malignant potential.

TABLE 6
17 cases (3.4% of independent validation cohort) misclassified by the 35-GEP.

| | | | | Growth pattern | | Breslow | | |
|---|---|---|---|---|---|---|---|---|
| | 35-GEP | | | submitted by | | thickness | | |
| Sample # | result | Sex | Age | dermatopathologist | Atypia | (mm) | Ulceration | Location |
| 91 | malignant | M | 2 | Spitz Nevus | None | NA | NA | Extremities |
| 315 | malignant | M | 3 | Spitz Nevus, Compound | None | NA | NA | Extremities |
| 95 | malignant | M | 6 | Spitz Nevus | None | NA | NA | Head/Neck |
| 98 | malignant | M | 6 | Spitz Nevus | None | NA | NA | Head/Neck |
| 129 | malignant | M | 8 | Benign Pigmented | None | NA | NA | Extremities |
| | | | | Spindle-Cell Nevus of | | | | |
| | | | | Reed (Variant of Spitz) | | | | |
| 318 | malignant | M | 8 | Pigmented Spindle Cell | None | NA | NA | Head/Neck |
| | | | | Variant of Spitz's Nevus | | | | |
| 297 | malignant | M | 33 | Junctional Melanocytic | Mild | NA | NA | Back |
| | | | | Nevus | | | | |
| 266 | malignant | F | 36 | Junctional Spitz Nevus | None | NA | NA | Extremities |
| 300 | malignant | M | 37 | Compound Dysplastic | Mild | NA | NA | Back |
| | | | | Melanocytic Nevus | | | | |
| 69 | malignant | F | 42 | Combined Blue and | None | NA | NA | Abdomen/Chest |
| | | | | Intradermal Nevus | | | | |
| 46 | malignant | F | 43 | Junctional Dysplastic | Moderate | NA | NA | Back |
| | | | | Nevus | | | | |
| 138 | malignant | F | 55 | Benign Melanocytic | None | NA | NA | Acral |
| | | | | Nevus | | | | |
| 324 | malignant | F | 56 | Compound Melanocytic | None | NA | NA | Back |
| | | | | Nevus | | | | |
| 323 | malignant | F | 58 | Intradermal Melanocytic | None | NA | NA | Extremities |
| | | | | Nevus | | | | |
| 313 | malignant | M | 61 | Junctional Melanocytic | Moderate | NA | NA | Back |
| | | | | Nevus | | | | |
| 566 | benign | F | 63 | Melanoma in situ | NA | NA | No | Extremities |
| 404 | benign | M | 83 | Nodular melanoma | NA | 4.0 | No | Head/Neck |

NA—not addressed.

### Example 4: 35-GEP Intermediate-Risk Zone

Given the potential biological transition of melanocytic lesions from a benign to a malignant state, the 35-GEP profile was developed to identify lesions with an intermediate-risk of malignancy. Inclusion of a wide variety of subtypes in the study improved the classification of lesions as benign or malignant and led to a restricted intermediate-risk zone. A total of 96.4% of cases had a definitive benign or malignant test result and only 3.6% (18/503) of cases were classified as intermediate-risk, including eight melanomas and ten benign nevi. Though a definitive benign or malignant result is advantageous for implementing patient management pathways, samples with probability scores in the intermediate-risk zone may be evolving, borderline, or atypical and warrant special consideration in terms of patient management with a focus on clinicopathologic correlation.

Dermatopathologists have a number of ancillary tools available to assist with the diagnosis of pigmented lesions, yet there is a substantial amount of diagnostic discordance that may potentially lead to overtreatment of patients with benign lesions and undertreatment of patients with melanoma. The 35-GEP test to distinguish benign from malignant pigmented lesions was developed to improve diagnostic accuracy and reduce diagnostic uncertainty for difficult-to-diagnose cases. A cross-study analysis shows that in an independent validation cohort of 503 benign lesions and melanomas the 35-GEP test demonstrated improved accuracy compared to other diagnostic tools based on their primary validation studies (Table 7).

TABLE 7
Comparison of 35-GEP to currently available ancillary tests.

| | # of | Type of | | | Technical | Nevi subtypes | Melanoma subtypes |
|---|---|---|---|---|---|---|---|
| Study | cases | test | Sensitivity | Specificity | failure | included | included |
| Current | 503 | 35-GEP | 99.1% | 94.3% | 3.4% | Blue, common, | Acral, |
| study | | | | | | deep penetrating, | desmoplastic, |
| | | | | | | dysplastic, Spitz | lentiginous, lentigo |
| | | | | | | | maligna, in situ, |
| | | | | | | | nevoid, nodular, |
| | | | | | | | superficial |
| | | | | | | | spreading, Spitzoid |
| (Clarke | 437 | 23-GEP | 94.0% | 90.0% | 14.7% | Blue, common, | Acral, lentigo |
| et al., | | | | | | dysplastic, Spitz | maligna, nodular, |
| 2015) | | | | | | | superficial |
| | | | | | | | spreading |
| (Clarke | 736 | 23-GEP | 91.5% | 92.5% | NA | Not reported | Acral, lentigo |
| et al., | | | | | | | maligna, nodular, |
| 2017a) | | | | | | | superficial |
| | | | | | | | spreading |
| (Gerami | 196 | FISH^{#} | 86.7% | 95.4% | NA | Acral, blue, | Not reported |
| et al., | | | | | | common, | |
| 2009) | | | | | | dysplastic, Spitz | |
| (Gerami | 233 | FISH^{#} | 83.0% | 94.0% | NA | Blue, common, | Acral, lentigo |
| et al., | | | | | | dysplastic, Spitz | maligna, nodular, |
| 2010) | | | | | | | superficial |
| | | | | | | | spreading |
| (Lezcano | 400 | PRAME | 84.7%^{&} | 99.2%^{&} | NA | Common, | Acral, cutaneous |
| et al., | | IHC | | | | dysplastic, Spitz | paramucosal, |
| 2018) | | | | | | | desmoplastic, |
| | | | | | | | lentigo maligna, |
| | | | | | | | nevoid, nodular, |
| | | | | | | | superficial |
| | | | | | | | spreading |
| (Lezcano | 110 | PRAME | 75.0% | 98.8% | NA | Blue, common, | Acral, malignant |
| et al., | | IHC | | | | deep penetrating, | melanoma, nevoid, |
| 2020) | | | | | | dysplastic, Spitz | Spitzoid |

^{#}6p25, Cep 6, 6q23, and 11q13.
^{&}Calculated from the data reported in the manuscript.
NA—not addressed.

Unlike FISH, CGH or IHC, gene expression profiling captures transcriptomic events within the lesion and the surrounding tissue, allowing for a more comprehensive assessment of the biological changes that are associated with the transition to a malignant phenotype. IHC generally allows for evaluation of changes in the expression of a single biomarker at the protein level, which can be limited by subjective quantification systems. PRAME IHC has been reported as a reliable method to distinguish benign from malignant pigmented lesions, however, ˜14% of nevi can have some staining for PRAME and the interpretation of positive staining (4+, ≥76% of immunoreactive tumor cells are PRAME positive) can be subjective. Thus, PRAME IHC requires further validation for widespread clinical use due to the potential for misdiagnosis of benign lesions as malignant. In the current study, PRAME expression did not improve diagnostic accuracy above the results reported for the 35-GEP (data not shown).

In this study, the 35-GEP reliably diagnosed 96.4% of benign and malignant lesions. In cross-study comparison (Table 7), the 35-test out-performed a 23-GEP diagnostic test with previously reported accuracy metrics for unequivocal samples ranging from 91.5-94% for sensitivity, 90.0-92.5% for specificity, technical failures in 14.7%, moreover, ˜15% of diagnostically concordant (i.e. 3 out of 3) cases could not be classified as benign or malignant (Clarke et al., 2017a, 2015). By comparison, the 35-GEP test demonstrated sensitivity (99.1%) and specificity (94.3%) in all ages and 99.1% sensitivity and 96.2% specificity in patients >18 years old, a low number of technical failures (3.4%), and no more than 3.8% of cases received an intermediate-risk result. The improved classification of lesions compared to that of the 23-GEP test is likely due to implementation of highly sophisticated modeling (neural networks) that resulted in two algorithms with 32 diagnostic and 3 control genes, the inclusion of samples with different growth patterns (total of nine melanoma subtypes and nine benign subtypes) in the training cohort as well as incorporation of lesions with 2/3 concordance.

Data supporting the utility of the 23-GEP test in ambiguous or diagnostically discordant lesions is limited. Recently, sensitivity of 90.4% and specificity of 95.5% was reported for 125 ‘uncertain’ cases, however, the definition of uncertainty was broad and included lesions as discordant if a differing diagnosis was received from just 1 of 7 dermatopathologists reviewing the cases. In this study, cases with concordance for 2 of 3 reviewing dermatopathologists were included in this independent 35-GEP validation. The 35-GEP was developed and validated using fully concordant lesions and a small set of ‘borderline’ cases, where no more than 1 out of 3 dermatopathologists indicated ‘unknown malignant potential’ as a diagnosis. Since the 35-GEP will be most likely used in difficult-to-diagnose lesions, inclusion of 2/3 concordant cases to capture differentially expressed genes from those histopathologically challenging cases were factored into the neural network configuration during the test development. With the improved accuracy metrics and substantially reduced intermediate-risk zone, dermatopathologists can expect a definitive result from the 35-GEP test in ≥95% of lesions submitted for testing. Improved test characteristics for the disambiguation of pigmented lesions will help refine guidelines for when to utilize GEP in the diagnosis of challenging pigmented lesions.

Although the vast majority of cases tested by the 35-GEP will have a definitive score of benign or malignant risk potential, 3.6% of cases fell into an intermediate-risk zone reflective of a molecular biology characteristic of both benign and malignant lesions. Though the prevalence is not known, evidence that there is a true ‘transition’ zone for pigmented lesions is mounting. Thus, interpretation of an intermediate-risk result of the 35-GEP should be considered in the context of other clinicopathological information. Specifically, in cases with an intermediate-risk score, it would be of great diagnostic importance to exclude the possibility of sampling error and ensure that the entire clinical lesion has been evaluated by routine histopathology. Unfortunately, up to ⅓ of nevi transition to melanoma, so there is a subset of lesions that may be clinically identified during this progression. In addition, there are atypical melanocytic proliferations (AMPs) that never evolve to full malignancy despite metastasis to regional lymph nodes. The spectrum of outcomes for these lesions warrants special consideration in clinical management. Clinical management of AMPs varies as there are no official guidelines governing their treatment, but common practice is definitive surgical treatment with removal of lesion with the margin of normal skin. In addition, the use of 35-GEP can provide the dermatologist and/or patient with treatment options to cover the most severe of diagnoses, including a diagnosis of melanoma. Studies are underway in a true AMP population with known outcomes.

The 35-GEP test performed equally well in nevi and melanomas with different growth patterns. For instance, classification of lentigo maligna, nodular and superficial spreading melanomas was concordant with dermatopathologic diagnosis as were blue and common nevi (compound, intradermal and junctional), along with dysplastic nevi with varying degree of atypia with only a small percentage receiving an intermediate-risk result. Further studies to increase the number of samples in subtypes that were not represented in large enough numbers are underway. The Spitz subtype is particularly challenging and thus far all available ancillary tests have had limitations in sensitivity and specificity.

For the dermatologist, metastatic risk assessment is critical for guiding appropriate patient management following a melanoma diagnosis. A prognostic 31-GEP test has been validated to determine individualized 5-year risk for recurrence, metastasis and melanoma-specific survival. Based on accuracy metrics and multivariate models demonstrating that the test is an independent and significant risk-prediction tool, the value of the 31-GEP result as an adjunct to current staging factors has been recognized by the National Comprehensive Cancer Network. Thus, patients diagnosed with malignant lesions have effective prognostic tools and contemporary therapies, with demonstrated improved outcomes, at their disposal.

Given the availability of the prognostic 31-GEP test for cutaneous melanoma, the 35-GEP test was developed to enhance the diagnosis of benign nevi and melanomas by providing dermatopathologists with an objective ancillary tool to aid in their diagnosis of difficult-to-diagnose pigmented lesions. Clinically implemented GEP tests for diagnostically challenging melanocytic lesions have demonstrated high impact on utility for guiding decision-making.

Although not the focus of this study, assessment of 35-GEP clinical utility, as well as correlation of test results with outcomes, is underway. In the zone of significant uncertainty, the high accuracy metrics of the test might increase confidence level in diagnosis to dermatopathologists and dermatologists, while providing assurance to the patients. The test also provided a definitive result for 96.4% of the lesions in the validation study, offering an opportunity to reduce the uncertainty associated with pigmented lesions and promote more definitive management of patients by dermatologists. An ancillary test with the characteristics reported here could impact expenditure on over-diagnoses by decreasing unnecessary surgeries, imaging and follow-up while more appropriately allocating healthcare resources to those lesions where metastatic risk is identified.

All references cited in this application are expressly incorporated by reference herein.

What is claimed is:

1. A method for treating a patient with a skin lesion, the method comprising:
(a) obtaining a diagnosis identifying the skin lesion as a malignant lesion, an intermediate risk lesion, or a benign lesion in a sample from the skin lesion of the patient, wherein the diagnosis was obtained by:
(1) determining the expression level of at least 12 genes in a gene set; wherein the at least 12 genes in the gene set are selected from:
ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1;
(2) comparing the expression levels of the at least 12 genes in the gene set from the sample to the expression levels of the at least 12 genes in the gene set from a predictive training set to generate a probability score;
(3) providing an indication as to whether the skin lesion is a malignant lesion, an intermediate risk lesion, or a benign lesion based on the probability score generated in step (2); and
(4) identifying that the skin lesion is a malignant lesion based on the probability score; and
(b) administering to the patient an aggressive treatment when the determination is made in the affirmative that the patient has a skin lesion that is a malignant lesion.

2. The method of claim 1, further comprising performing a resection of the skin lesion when the determination is made in the affirmative that the patient has a skin lesion that is a malignant lesion.

3. The method of claim 1, wherein the expression level of each gene in a gene set is determined by reverse transcribing the isolated mRNA and measuring a level of fluorescence for each gene in the gene set by a nucleic acid sequence detection system following RT-PCR.

4. The method of claim 1, wherein the sample from the skin lesion is obtained from a formalin-fixed, paraffin embedded sample.

5. The method of claim 1, wherein the probability score is between 0 and 1, and wherein a value of 1 indicates a higher probability of a malignant lesion than a value of 0.

6. The method of claim 1, wherein the probability score has a sensitivity of at least 90%, 91%, 92%, 93%, 94%, 95%, 96%, 97%, 98%, or 99%, and/or has a specificity of at least 90%, 91%, 92%, 93%, 94%, 95%, 96%, 97%, 98%, or 99%.

7. The method of claim 1, wherein the probability score has a positive predictive value (PPV) of at least 90%, 91%, 92%, 93%, 94%, 95%, 96%, 97%, 98%, or 99%, and/or has a negative predictive value (NPV) of at least 90%, 91%, 92%, 93%, 94%, 95%, 96%, 97%, 98%, or 99%.

8. The method of claim 1, wherein the gene set further comprises at least one control gene, wherein the at least one control gene is selected from the group consisting of FXR1, HNRNPL, and YKT6.

9. The method of claim 8, wherein the control genes are FXR1, HNRNPL, and YKT6.

10. A method of treating a patient with a skin lesion, the method comprising administering an aggressive cancer treatment regimen to the patient,
wherein the patient has a skin lesion identified as a malignant lesion as generated by comparing the expression levels of at least 12 genes wherein the at least 12 genes are selected from ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1, from the skin lesion with the expression levels of the same at least 12 genes are selected from ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1 from a predictive training set.

11. The method of claim 10, wherein the skin lesion is determined to be a malignant lesion, an intermediate risk lesion, or a benign lesion.

12. The method of claim 10, wherein the gene set further comprises at least one control gene, wherein the at least one control gene is selected from the group consisting of FXR1, HNRNPL, and YKT6.

13. The method of claim 12, wherein the control genes are FXR1, HNRNPL, and YKT6.

14. A kit comprising primer pairs suitable for the detection and quantification of nucleic acid expression of at least 12 genes, wherein the at least 12 genes are selected from: ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1.

15. The kit of claim 14, wherein the kit further comprises primer pairs suitable for the detection and quantification of nucleic acid expression of at least one control gene, wherein the at least one control gene is selected from the group consisting of FXR1, HNRNPL, and YKT6.

16. The kit of claim 15, wherein the kit comprises primer pairs for 3 control genes, wherein the 3 control genes are FXR1, HNRNPL, and YKT6.

17. A method for diagnosing a skin lesion from a patient as a malignant lesion, an intermediate risk lesion, or a benign lesion, the method comprising:
(a) obtaining a sample from the skin lesion from the patient and isolating mRNA from the sample;
(b) determining the expression level of at least 12 genes in a gene set; wherein the at least 12 genes in the gene set are selected from: ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1;
(c) comparing the expression levels of the at least 12 genes in the gene set from the sample to the expression levels of the at least 12 genes in the gene set from a predictive training set to generate a probability score; and
(d) providing an indication as to whether the skin lesion is a malignant lesion, an intermediate risk lesion, or a benign lesion, based on the probability score generated in step (c).

18. The method of claim 17, wherein the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR).

19. The method of claim 17, wherein the sample from the skin lesion is obtained from a formalin-fixed, paraffin embedded sample.

20. The method of claim 17, wherein the probability score is between 0 and 1, and wherein a value of 1 indicates a higher probability of a malignant lesion than a value of 0.

21. The method of claim 17, wherein the probability score has a sensitivity of at least 90%, 91%, 92%, 93%, 94%, 95%, 96%, 97%, 98%, or 99%, and/or has a specificity of at least 90%, 91%, 92%, 93%, 94%, 95%, 96%, 97%, 98%, or 99%.

22. The method of claim 17, wherein the probability score has a positive predictive value (PPV) of at least 90%, 91%, 92%, 93%, 94%, 95%, 96%, 97%, 98%, or 99%, and/or has a negative predictive value (NPV) of at least 90%, 91%, 92%, 93%, 94%, 95%, 96%, 97%, 98%, or 99%.

23. The method of claim 17, further comprising identifying the skin lesion as a malignant lesion based on the probability score, and administering to the patient an aggressive tumor treatment.

24. The method of claim 17, wherein the gene set further comprises at least one control gene, wherein the at least one control gene is selected from the group consisting of FXR1, HNRNPL, and YKT6.

25. The method of claim 24, wherein the control genes are FXR1, HNRNPL, and YKT6.

26. A method for diagnosing a skin lesion from a patient as a malignant lesion, an intermediate risk lesion, or a benign lesion, the method comprising:
(a) obtaining a sample from skin lesion from the patient and isolating mRNA from the sample;
(b) determining the expression level of at least 12 genes in a gene set; wherein the at least 12 genes in the gene set are: ABLIM1, ANXA8L1, ATP6V0E2, BAP1, BCL2A1, BTG1, CLCA2, CST6, CSTA, CXCL14, DCT, DSP, DUSP4, GATA3, GJA1, GPR143, HAL, MGP, KLF5, KRT2, KRT17, NES, PPL, RPL37A, PTN, RPS16, S100A8, S100A9, SAP130, SFN, TP63, and WIPI1; and
(c) providing an indication as to whether the skin lesion is a malignant lesion, an intermediate risk lesion, or a benign lesion, based on the expression level of the at least 12 genes generated in step (b).

27. The method of claim 26, wherein the expression level of each gene in the gene set is determined by reverse transcribing the isolated mRNA into cDNA and measuring a level of fluorescence for each gene in the gene set by a nucleic acid sequence detection system following Real-Time Polymerase Chain Reaction (RT-PCR).

28. The method of claim 26, wherein the sample from the skin lesion is obtained from formalin-fixed, paraffin embedded sample.

29. The method of claim 26, wherein the gene set further comprises at least one control gene, wherein the at least one control gene is selected from the group consisting of FXR1, HNRNPL, and YKT6.

30. The method of claim 29, wherein the control genes are FXR1, HNRNPL, and YKT6.
